(19) United States
(12) Patent Application Publication
(10) Pub. No.: US 2022/0168060 A1
(43) Pub. Date: Jun. 2, 2022

(54) DISPOSABLE SURGICAL DRAPE

(71) Applicant: ART, LIMITED, Grand Cayman (KY)

(72) Inventor: Takayuki Akahoshi, Tokyo (JP)

(21) Appl. No.: 17/441,396

(22) PCT Filed: Mar. 19, 2020
(86) PCT No.: PCT/US2020/023492

§ 371 Date: Sep. 21, 2021

Related U.S. Application Data
(60) Provisional application No. 62/821,554, filed on Mar. 21, 2019.

(51) Int. Cl. A61B 46/20

(52) U.S. Cl. CPC A61B 46/20; A61B 2046/205

(57) ABSTRACT

A disposable surgical drape is provided for use while performing surgical procedures on an eyes of a patient, or other surgical procedures. The surgical drape comprises a base sheet, defining first and second surgical apertures, for attachment to a patient, and at least one protective patch which can be applied over one of the surgical apertures after the drape has been positioned on the patient. The protective patch defines a slit through which the procedure can be performed, while the other surgical aperture remains open and unobstructed to minimize any patient claustrophobia. After the procedure is performed on one eye of the patient, the protective patch can be removed, and a fresh protective patch applied to the other surgical aperture to permit the procedure to be performed on the other eye of the patient.

### FIELD OF THE INVENTION

The present invention relates generally to the field of surgery including ophthalmic surgery, and more particularly, to a disposable surgical drape for use during surgery of the human eye, in particular bilateral surgery, as well for use during other surgical procedures. The surgical drape comprises a base sheet, defining first and second surgical apertures, for attachment to a patient, and at least one protective patch which can be applied over one of the surgical apertures after the drape has been positioned on the patient.

### BACKGROUND OF THE INVENTION

When the eye becomes aged, diseased, or injured it may be necessary to remove the natural lens of the eye. Such removal is common for cataract surgery, in which a lens that has become clouded is removed. The removal of the natural lens of the eye may result in the loss or alteration of focused vision of a patient. Eyeglasses, contact lenses, or the implantation of an artificial lens may be necessary to restore the vision of the patient.

During the removal of the natural lens of the eye, a physician will typically make one or more small incisions in the eye and insert a vibratory needle into the incision. The needle may be vibrated at ultrasonic speeds in torsional, longitudinal, elliptical, or blended modes in order to dismember the lens in a process known as phacoemulsification. The needle typically contains one or more aspiration passages for removing the broken lens particles from the eye. Lasers may also be used to dismember the lens.

This surgical process is typically performed with an ophthalmic drape being placed over the eye of the patient. The ophthalmic drape has one aperture for being placed over the eye upon which surgery will occur, The drape has an aperture for insertion of the surgical equipment and may have an adhesive to secure the drape to the patient's skin. A topical or local anesthetic is applied to the eye. However, general anesthesia may be required for some patients who cannot remain still for the surgical procedure or for some patients who may suffer from claustrophobia. The obstructed vision of the eye which is not being operated upon may further exacerbate such claustrophobia. General anesthetic can add substantial expense to the surgical operation and may further present risks to the health of the patient, when compared to local anesthetic.

### SUMMARY OF THE INVENTION

The present invention is directed to a disposable surgical drape with a wide range of potential applications. The inventor of the present invention has discovered the present drape can made particularly suitable for ophthalmic surgical procedures, in particular a bilateral procedure, and can be configured for minimizing the claustrophobia of a patient undergoing ophthalmic surgery. Notably, the drape includes a base sheet having a pair of surgical apertures and having an adhesive, preferably a pressure-sensitive adhesive, to facilitate securement of the base sheet to the patient. The drape includes one or more protective patches, also provided with an adhesive, preferably a pressure-sensitive adhesive, for sequentially covering each one of the surgical apertures during the procedure. Each protective patch defines a slit through which the surgical procedure can be performed, while the other surgical aperture remains open and unobstructed to minimize or prevent patient claustrophobia. After the procedure is performed on one eye of the patient, the protective patch can be removed, and a fresh protective patch may be applied to the other surgical aperture to permit the surgical procedure to be performed on the other eye of the patient.

In accordance with the present invention, a disposable surgical drape is provided, which is particularly useful for ophthalmic surgery, including a bilateral procedure. The drape has a base sheet of thin, flexible material, The base sheet has an upper surface, a lower surface for contacting a patient, and at least one edge.

To facilitate surgical use, the base sheet of the present surgical drape includes first and second surgical apertures formed through the base sheet, with the surgical apertures each having a length, and with surgical apertures for being located proximal to first and second eyes of the patient. An attachment zone formed on the base sheet lower surface, comprising pressure-sensitive adhesive, is located proximal to the first and second surgical apertures, preferably at least partially surrounding the surgical apertures. The attachment zone thus facilitates attachment of the base sheet lower surface to the patient to secure the base sheet to the patient in the desired position for performing the surgical procedure. In the preferred embodiment, the attachment zone extends completely about the first and second surgical apertures on the lower surface of the base sheet.

In accordance with the present invention, the present surgical drape further includes at least one protective patch for positioning at and substantially covering one of the surgical apertures to protect the eye of the patient. Each protective patch has the form of a polymeric sheet having pressure-sensitive adhesive applied to one surface thereof, and defines an elongated slit through which the surgical procedure is performed. The pressure sensitive adhesive on each of the protective patches is preferably provided on the periphery thereto, to thereby define an adhesive-free region surrounding the slit defined by the protective patch.

Thus, after positioning of the base sheet on the patient, with the first and second apertures respectively aligned with the eyes of the patient, one of the protective patches can be applied over one of surgical apertures, and removably secured to the base sheet with the pressure-sensitive adhesive provided on the protective patch. After the surgical procedure is performed through the protective patch, the patch can be removed and discarded, and a fresh protective patch applied to the other surgical aperture. The surgical procedure can them be performed on the other eye of the patient.

In the illustrated preferred embodiment of the invention, the surgical drape further includes at least one detachable pouch positioned in proximity to one of the first and second apertures defined by the base sheet. The detachable pouch preferably defines an opening facing toward that one of the surgical apertures to which it is positioned in proximity. In the preferred form, the disposable surgical drape includes a pair of the detachable pouches positioned in respective proximity to the first and second surgical apertures defined by the base sheet.

To facilitate efficient use, the base sheet of the surgical drape is folded at lateral margins thereof so that the first and second surgical apertures remain uncovered by the lateral margins,

In accordance with the present invention, a method of covering a patient for bilateral eye surgery comprises the steps of providing a surgical drape comprising a base sheet of flexible material having an upper surface, and a lower surface for contacting a patient, with first and second surgical apertures formed through the base sheet. The surgical apertures each have a length, with the surgical apertures being positionable proximal to (aligned overtop of) the first and second eyes of the patient. The surgical drape further comprises an attachment zone formed on the base sheet lower surface, with the attachment zone being located proximal to the first and second surgical apertures, preferably at least partially surrounding the surgical apertures for attaching the base sheet lower surface to the patient to fix at least a portion of the base sheet with respect to the patient.

The present method further comprises providing at least one protective patch for positioning at one of the surgical apertures to protect the eye of the patient. Each protective patch comprises a polymeric sheet having pressure-sensitive adhesive applied to one surface thereof, and defines an elongated slit. After positioning of the base sheet on the patient with the first and second apertures respectively aligned with the eyes of the patient, with the attachment zone secured to the patient; the protective patch is applied over one of the surgical apertures, with the protective patch removably secured to the base sheet with the pressure-sensitive adhesive.

In the preferred practice, the present method includes providing at least one detachable pouch on the base sheet, positioned in proximity to one of the first and second surgical apertures. Each pouch is removably secured to the base sheet with the pressure-sensitive adhesive and is designed to catch any overflow of balanced salt solution that may not be aspirated into the surgical devices used in the surgical procedure.

The present method contemplates that after the surgical procedure is performed on the first eye of the patient, the protective patch and the detachable pouch for that eye are then removed from the base sheet. Thereafter, another protective patch and another pouch may applied to the base sheet at the other surgical aperture, and at the other eye of the patient.

Another feature of the present invention is to provide a low-cost, disposable surgical drape for the reduction and/or minimization of claustrophobia in a patient.

In another aspect of the present invention, a folded surgical drape is provided to facilitate positioning of the drape on a patient, and subsequent spreading of the drape over the patient.

Other features and advantages of the present invention will be better understood with reference to the accompanying figures and detailed description.

### BRIEF DESCRIPTION OF THE DRAWINGS

FIG. 1 is a diagrammatic, top plan view of a disposable surgical drape embodying the principles of the present invention shown in both an unfolded and folded configuration;

FIG. 2 is a diagrammatic, top plan view of a pair of protective patches which can be selectively secured to a base sheet of the disposable surgical drape according to the present invention;

FIG. 3 is a diagrammatic, bottom plan view of the present disposable surgical drape of FIG. 1 showing an attachment zone for removably securing the drape on a patient;

FIG. 4 is a diagrammatic top plan view of one of the protective patches shown in FIG. 2;

FIG. 5 is a diagrammatic bottom plan view of one of the protective patches shown in FIG. 2, illustrating removal of two embodiments of a protective layer from pressure-sensitive adhesive provided on the protective patch;

FIG. 6 is a diagrammatic view illustrating the present surgical drape in a folded condition for unfolding and securement to a patient for the desired surgical procedure;

FIG. 7 is a diagrammatic view illustrating the present surgical drape in an unfolded condition and in position on a patient in preparation for the desired surgical procedure;

FIG. 8 is a diagrammatic view similar to FIG. 7, illustrating the present surgical drape during the surgical procedure on the first eye of the patient, and FIG. 8 shows that on protective patch has been applied to the base sheet of the drape substantially over the first eye of the patient and a pair of removable pouches have been applied to the base sheet of the drape at a location adjacent each eye to catch irrigation fluid;

FIG. 9 is a diagrammatic view similar to Fig, 8, illustrating completion of the surgical procedure on the first eye of the patient, and FIG. 9 shows the removal of the patch and pouch adjacent the first eye of the patient; and

FIG. 10 is a diagrammatic view similar to FIG. 9 illustrating the present surgical drape in use for performing the surgical procedure on the second eye of the patient, and FIG. 10 shows that on protective patch has been applied to the base sheet of the drape substantially over the second eye of the patient.

### DETAILED DESCRIPTION OF THE PREFERRED EMBODIMENTS

While the present invention is susceptible of embodiment in various forms, there is shown in the drawings and will hereinafter be described a presently preferred embodiment, with the understanding that the present disclosure is to be considered an exemplification of the invention, and is not intended to limit the invention to the specific embodiment illustrated.

The present disposable surgical drape is particularly suited for use in connection with ophthalmic surgical procedures, in particular a bilateral procedure. However, features of the surgical drape permit it be advantageously used in other procedures, including central venous catheterization (CVC), other catheterization procedures, anesthesia procedures, and other surgical procedures, as will be described.

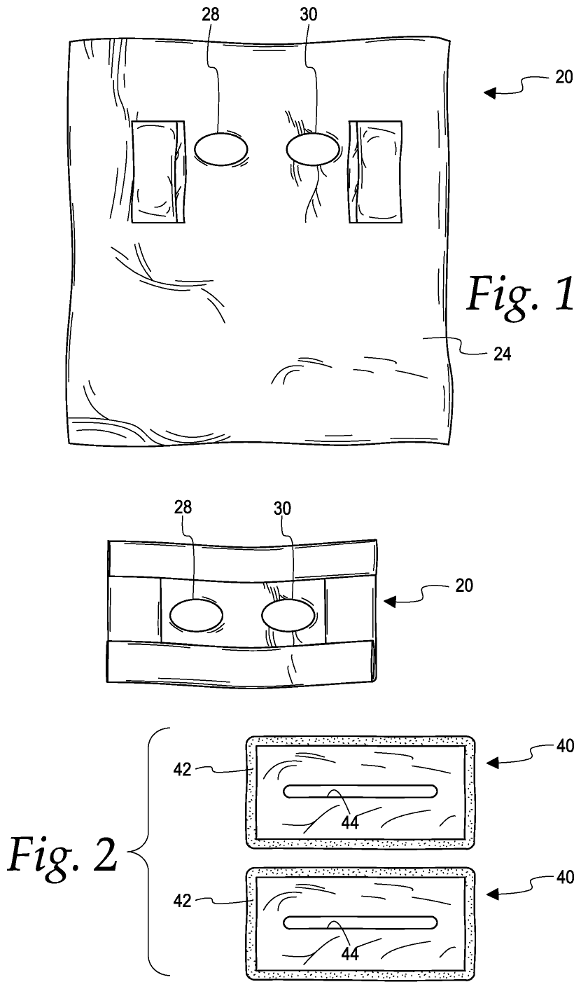

FIG. 1 is a top plan view of a disposable surgical drape 20, which is particularly useful for ophthalmic procedures, embodying the principles of the present invention. Drape 20 is formed from a generally rectangular base sheet 24 made from a thin, flexible material. Base sheet 24 can be made from tissue paper, textiles, fluid-permeable and non-fluid permeable polymers, and/or composites thereof. Preferably, the base sheet 24 is constructed from a light, water resistant paper that will be disposed of after a single use. In another presently preferred form, the base sheet 24 comprises a composite nonwoven SMS (spunbond-meltblown-spunbond) fabric. The drape 20 need not have a generally rectangular base sheet 24, but may have any of a variety of shapes, such as polygonal, circular, oval, or irregular shapes to conform to the area of the patient undergoing the surgical procedure.

The base sheet 24 has an upper surface (visible in FIG. 1), a lower surface (visible in FIG. 3), and an edge therebetween. The lower surface is positionable upon and in contact with the face of a patient undergoing an ophthalmic surgery.

As seen in FIG. 1, the base sheet 24 defines first and second surgical apertures 28, 30 extending through the base sheet 24 for being respectively positioned over the eyes of a patient during ophthalmic surgery. The apertures 28, 30 are typically initially formed in the base sheet 24, cut from the base sheet 24, or are subsequently created by tearing or rupturing of the base sheet 24 along a weakened or perforated area. The apertures 28, 30 can also be formed by incision or piercing of the base sheet 24 by a surgical instrument. The apertures 28, 30 are typically in the shape of a circle, oval, or elongate slot shape. Preferably, the apertures 28, 30 are large enough to provide the patient with an unobstructed view of his or her surroundings to minimize the likelihood of claustrophobia.

As can be seen in FIG. 3, a bottom plan view of the drape 20 showing the lower surface of the base sheet 24 thereof, drape 20 further has an attachment zone 32, preferably comprising a pressure-sensitive adhesive at least partially surrounding the apertures 28,30 so that the base sheet 24 can be removably affixed and secured to the face of the patient during the surgical procedure or procedures. In the preferred form, the attachment zone extends completely about the apertures 28, 30. A protective layer 34 is removably secured to the pressure-sensitive adhesive of the attachment zone 32 to maintain the adhesive in a fresh condition prior to use. While the attachment zone 32 is shown fully surrounding the surgical apertures 28, 30, the attachment zone 32 may alternatively be provided only partially surrounding the surgical apertures 28, 30, or may be located elsewhere on the base sheet lower surface.

Notably, the provision of first and second surgical apertures 28, 30 facilitates the surgical procedure, and desirably minimizes any claustrophobia that the patient might experience, since one eye can remain uncovered while the surgical procedure is performed on the other eye. The inventor has found that providing a pair of surgical apertures, used in sequence such as during a bilateral procedure, can desirably minimize the anxiety of claustrophobic patients because the vision of the second eye is not obscured by the base sheet 24 during the operation. The provision of such stereoscopic vision to the patient can provide a low-cost alternative to general anesthetic and can reduce the movement of an anxious, claustrophobic patient.

Another important feature of the drape 20 is that the eye position can be better controlled by the fixation light seen by the non-operated eye. In the prior art, the conventional fixing light was incorporated into a microscope to control the operated eye position during the surgery. However, due to the bright illumination of the microscope and poor visual acuity of the operated eye, such a fixing light was not ideal. When the non-operated eye can see through the non-surgical one of the apertures, the non-operated eye can be effective in leading the eye position during surgery.

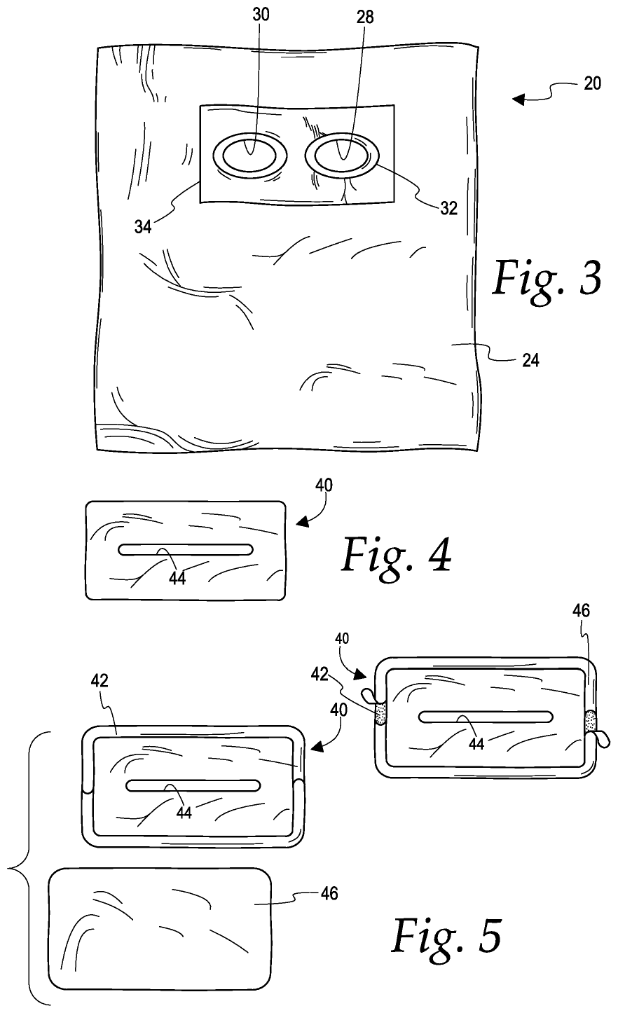

In accordance with the present invention, and with reference to FIGS. 2, 4, and 5, the present surgical drape 20 further includes at least one protective patch 40 for positioning at and covering one of the surgical apertures 28, 30 to protect the eye of the patient. Preferably, the drape 20 includes a pair of the protective patches 40, each of which can be used once for the procedure to be performed on each of the eyes of the patient.

Each protective patch 40 comprising a polymeric sheet having pressure-sensitive adhesive 42 (visible in FIG. 5) applied to one surface thereof, and defines an elongated slit 44 through which the surgical procedure is performed. As illustrated in FIG. 2, the pressure-sensitive adhesive on each of protective patches 40 is preferably provided on the periphery thereof, to thereby define an adhesive-free region surrounding the slit 44 defined by the protective patch. Preferably, as shown in FIG. 5, each protective patch 40 is provided with a removable protective layer 46 to maintain the pressure-sensitive adhesive 42 in a fresh condition prior to use. FIG. 5 illustrates two alternative embodiments of the protective layer 46 in the form of a layer that extends across the entirety of the patch 40 and in the form of a removable strip of material that extends only around the periphery of the patch 40 to cover the adhesive 42.

As can further be seen in FIG. 7, the drape 20 further preferably includes at least one, and more preferably a pair of, detachable pouches or compartments 52 on the upper surface of base sheet 24. Each pouch 52 is preferably located proximal to a respective one of the surgical apertures 28, 30, with an opening of each pouch facing toward the respective surgical aperture. Each pouch 52 is designed to catch any overflow of balanced salt solution that may not be aspirated into the surgical devices used in the surgical procedure. Typically, the balanced salt solution will be applied to the eye undergoing surgery as an irrigation solution to maintain pressure within the eye, The balanced salt solution is then typically aspirated through one or more lumens for sanitary disposal. Excess balanced salt solution that is not aspirated into such lumens may collect on the base sheet 24. The pouches 52 help to eliminate or at least control the accumulation of balanced salt solution. Alternatively, the pouches 52 can retain various surgical tools, equipment, and/or supplies when the drape 20 is draped over and attached to the face of the patient. The pouches 52 may be integrally formed with the drape via heat welding, adhesive, or stitching to the upper surface of the base sheet 24. Preferably, the pouches 52 are removable to reduce the weight of the base sheet 24 on the face of the patient to reduce impediments to breathing which could increase a sense of claustrophobia. In one preferred form of the invention, the pouch 52 would be applied just prior to the operation on a particular adjacent eye of the patient, then the pouch 52 would be removed just after the operation.

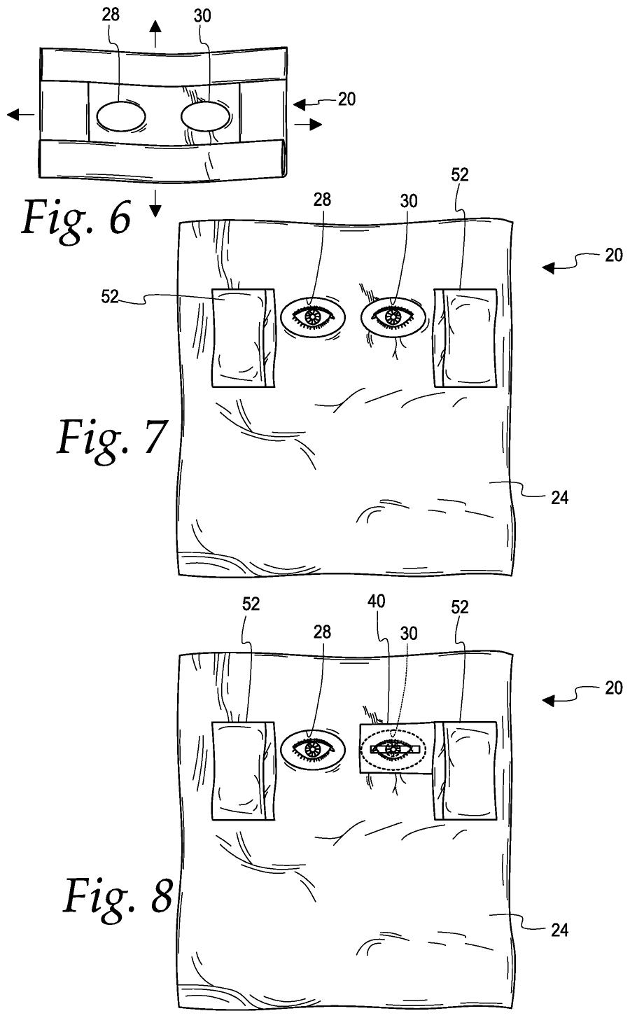

The method of use of the present ophthalmic drape 20 for covering a patient, such as for bilateral eye surgery, will now be described. In this regard, it will be noted that the drape 20 is preferably folded in a fashion (FIG. 6) such that lateral margins of the base sheet 24 are folded so that the first and second surgical apertures 28, 30 remain uncovered by the lateral margins. This facilitates placement of the drape 20 on a patient, and securement of the attachment zone 32 of the drape to the face of the patient so the desired surgical procedure can be performed.

After removal of protective layer 34 from the attachment zone 32 to expose the pressure-sensitive adhesive thereof, the surgical drape 20 is placed on the face of the patient and secured with the adhesive of the attachment zone. The lateral margins of the base sheet 24 can then be unfolded, with the drape 20 now in position with the lower surface of the base sheet 24 attached to the patient, as illustrated in FIG. 7. The patient is thus covered in the desired fashion for performing the surgical procedure. The surgical apertures 28, 30 are respectively positioned proximal to the of the patient.

As shown in FIG. 8, the present method next comprises providing at least one protective patch 40 for positioning at one of the surgical apertures 28, 30 to protect the eye of the patient. The protective layer 46 of the protective patch 40 is removed to expose the pressure-sensitive adhesive 42. After positioning of the base sheet 24 on the patient with the first and second apertures 28, 30 respectively aligned with the eyes of the patient, the protective patch 40 is applied over one of the surgical apertures (over aperture 30 in FIG. 8), with the protective patch 40 thus removably secured to the base sheet 24 with the pressure-sensitive adhesive. The surgical procedure is them performed through the slit 44 in the protective patch 42, while the other eye remains uncovered.

As noted, in the preferred form, at least one detachable pouch 52 is provided on the base sheet 24, positioned in proximity to one of the first and second surgical apertures 28, 30. Thus, as illustrated in FIG. 8, the pouch 52 is illustrated proximal to the aperture 30 to catch any overflow of balanced salt solution that may not be aspirated into the surgical devices used in the surgical procedure.

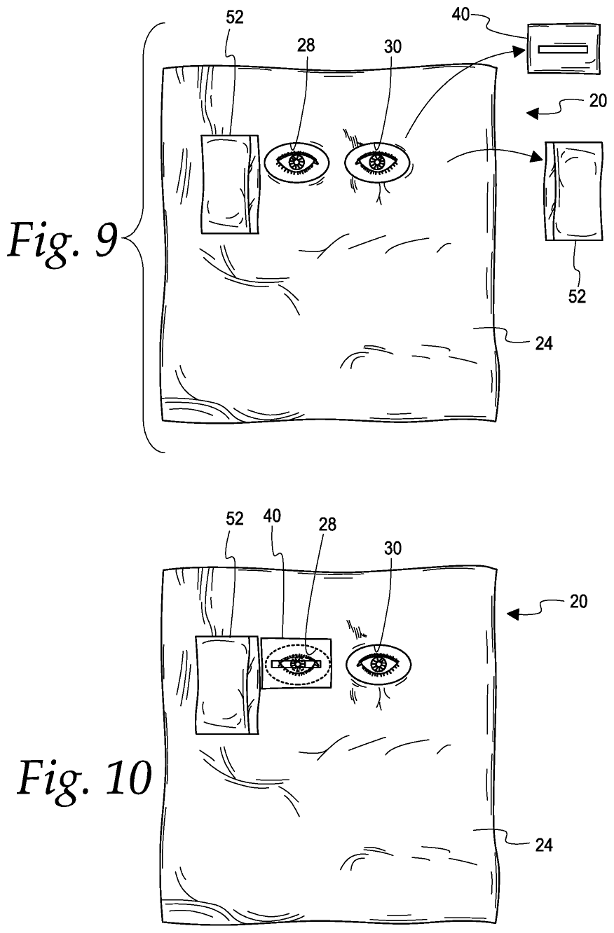

As shown in FIG. 9, the present method contemplates that after the surgical procedure is performed on the first eye of the patient, the protective patch 40 and detachable pouch 52 adjacent the first eye are removed from the base sheet 24 and discarded. Preferably, the adhesive of the protective patch 40 is selected such that it does not carry substantially any of the material of the drape base sheet 24 upon removal such that the drape base sheet 24 remains in place upon removal of the patch 40. Thereafter, as shown in FIG. 10, a second, fresh protective patch 40 is applied to the base sheet 24 over top of the other surgical aperture 28, at the other eye of the patient.

It is contemplated that the above-described embodiment may include a nose clip, or like spacing element, in order to provide height and clearance between the drape and the patient to facilitate convenient and comfortable breathing by the patient.

In the foregoing description, the present surgical drape has been described in connection with ophthalmic surgical procedures, but it is to be appreciated that the present surgical drape, including an attachment zone provided by a minimally-adhesive film, can be advantageously employed for other types of surgical procedures.

By way example, central venous catheterization (CVC) requires maximal sterile barrier precautions to prevent catheter-related bloodstream infection, which is associated with preventable morbidity and mortality. Disposable surgical drapes are recommended as a standard of care; however, those specifically meant for this purpose are neither freely available nor cost-effective. The present disposable drape can be advantageously employed for this purpose.

The present disposable surgical drape can be provided with an average size of 70 cm×70 cm, with surgical apertures 28. 30 appropriately sized, such as approximately 7 cm×9 cm.

The present disposable surgical drape provides an ideal option and ensures an optimum level of sterility. The adhesive area provided by attachment zone 32 acts to keep the drape in stable position, thereby avoiding the requirement of multiple drapes. Most current disposable drapes are made of low-lint and abrasion-resistant fabric and have level 4 liquid barrier performance because of their non-perforated design, and hence have poor absorbent quality as compared to the linen cloth drapes. The polyethylene drapes used in the absence of disposable drapes are completely non-absorbent and do not remain stable in place. The pouches 52 provided adjacent to the surgical apertures are designed to collect the irrigation fluid; during internal jugular vein annulation, the pouches can collect the trickle of blood that occurs after dilatation of the subcutaneous tract, preventing soiling and contamination of the neck and the shoulder area and the trickle over the drape down to the floor on the operator's feet.

The disposable surgical drape is more cost-effective as opposed to the one dedicated for CVC.

The present surgical drape can also be used successfully for subclavian, femoral and peripherally inserted central catheter line catheterizations. It can also be used for isolation during spinal or epidural anesthesia procedures and other regional blocks. During neuraxial blockade, the length of the transparent adhesive area should be placed along the length of the spine so that in case of difficulty one can easily have access to 2-3 interspinous spaces. The pouch 52 should be placed in the caudal direction. The best benefit is seen in an epidural procedure as on removing the Touhy needle after the insertion of the catheter; it is common to encounter a trickle of blood down the back soiling the operating table,

In summary, the present surgical drape provides a variety of desirable benefits. The drape is cost-effective, and can be used very efficiently to cover and protect the patient. Because the protective patches 40, with precut slits 44, are provided separately from the base sheet 24, the protective patches can be easily affixed to cover the eyelids of the patent, while the base sheet remains in position on the patient. The protective patches are not fixed to the large drape body.

Because both eyes of the patient can remain open, the present surgical drape desirably minimizes any claustrophobia the patient may experience. Having both eyes uncovered permits the uncovered eye to lead and fix the position of the eye on which the surgery is being performed.

The inventor has found that providing one or more protective patches 40 separately from the large base sheet 24 of the drape 20 not only reduces the cost to manufacture the drape 20 compared to drapes of the prior art, but is more easily and efficiently applied to the patient compared to drapes of the prior art. In one broad form of the present invention, the patch or patches 40 may be sold or otherwise provided separately from the base sheet 24 of the drape 20.

Use of the present surgical drape is also recommended for routine use for various procedures in anesthesia and the intensive care unit.

The illustrated preferred embodiments are included herein for descriptive purposes only and are not to be interpreted as limiting in any way of the broadest concepts of the present invention.

1. A disposable surgical drape (20), comprising:
a base sheet (24) of flexible material, said base sheet (24) having an upper surface, and a lower surface for contacting a patient;
first and second surgical apertures (28, 30) formed through said base sheet (24), said surgical apertures (28, 30) each having a length, said surgical apertures positionable proximal to first and second eyes of the patient;
an attachment zone (32) formed on said base sheet lower surface, said attachment zone (32) being located proximal to said first and second surgical apertures (28, 30), said attachment zone (32) at least partially surrounding said surgical apertures (28, 30), said attachment zone (32) permitting attachment of said base sheet lower surface to the patient to fix at least a portion of said base sheet (24) with respect to the patient; and
at least one protective patch (40) for positioning at one of said surgical apertures (28, 30) to protect the eye of the patient,
said at least one protective patch (40) comprising a polymeric sheet having pressure-sensitive adhesive applied to one surface thereof, and defining an elongated slit (44), whereby after positioning of said base sheet (24) on said patient with said first and second apertures (28, 30) respectively aligned with the eyes of the patient, said protective patch (40) can be applied over one of said apertures (28, 30) and removably secured to said base sheet (24) with said pressure-sensitive adhesive.

2. The disposable surgical drape (20) of claim 1, wherein said attachment zone (32) comprises a pressure-sensitive adhesive.

3. The disposable surgical drape (20) of claim 2, wherein said attachment zone (32) extends about said first and second surgical apertures (28, 30). Page 2 of 6

4. The disposable surgical drape (20) of claim 1, further comprising:
at least one detachable pouch (52) positioned in proximity to one of said first and second apertures (28, 30).

5. The disposable surgical drape (20) of claim 4, wherein said at least one detachable pouch (52) defines an opening facing toward said one of said first and second surgical apertures (28, 30).

6. The disposable surgical drape (20) of claim 1, further comprising:
a pair of said detachable pouches (52) positioned in respective proximity to said first and second surgical apertures (28, 30).

7. The disposable surgical drape (20) of claim 1, wherein
said pressure sensitive adhesive of said protective patch (40) is provided on a periphery thereof, to thereby define an adhesive-free region surrounding the slit (44) defined by the protective patch (40).

8. The disposable surgical drape (20) of claim 1, wherein said base sheet (24) is folded at lateral margins thereof so that said first and second surgical apertures (28, 30) remain uncovered by said lateral margins.

9. A method of covering a patient for bilateral eye surgery, comprising the steps of:
obtaining a surgical drape (20) comprising a base sheet (24) of flexible material having an upper surface, and a lower surface for contacting a patient, with first and second surgical apertures (28, 30) formed through said base sheet (24), said surgical apertures (28, 30) each having a length, said surgical apertures (28, 30) being positionable proximal to first and second eyes of the patient, said surgical drape (20) further comprising an attachment zone (32) formed on said base sheet lower surface, said attachment zone (32) being located proximal to said first and second surgical apertures (28, 30), said attachment zone (32) at least partially surrounding said surgical apertures (28, 30) for attaching said base sheet lower surface to the patient to fix at least a portion of said base sheet (24) with respect to the patient;
obtaining at least one protective patch (40) for positioning at one of said surgical apertures (28, 30) to protect the eye of the patient, said protective patch (40) comprising a polymeric sheet having pressure-sensitive adhesive applied to one surface thereof, and defining an elongated slit (44);
positioning said base sheet (24) on said patient with said first and second apertures (28, 30) respectively aligned with the eyes of the patient, with said attachment zone (32) secured to the patient; and
applying said protective patch (40) over one of said apertures (28, 30) and removably securing the protective patch (40) to said base sheet (24) with said pressure-sensitive adhesive.

10. A method of covering a patient for bilateral eye surgery in accordance with claim 9, further comprising the steps of obtaining at least one detachable pouch (52); and placing said detachable pouch (52) on said base sheet (24) in proximity to one of said first and second apertures (28, 30).

11. A method of covering a patient for bilateral eye surgery in accordance with claim 9, further comprising the steps of:
removing said protective patch (40) from said patient; and thereafter obtaining and applying another one of said protective patches (40) to the other eye of the patient.

12. A method of covering a patient for bilateral eye surgery in accordance with claim 10, further comprising the step of:
removing said detachable pouch (52) from said base sheet (24).

13. A protective patch (40) for use with a surgical drape (20) having at least one surgical aperture (28, 30), said patch (40) comprising:
a polymeric sheet having pressure-sensitive adhesive applied to one surface thereof, and defining an elongated slit (44), whereby said protective patch (40) is configured for being applied over said at least one surgical aperture (28, 30) of said drape (20).

14. The protective patch (40) of claim 13 wherein said pressure-sensitive adhesive is selected such that said patch (40) is removably secured to said drape (20) with substantially no material of said drape (20) removed with the removal of said patch (40).

15-26. (canceled)
